(19) United States
(12) Patent Application Publication
(10) Pub. No.: US 2011/0130695 A1
(43) Pub. Date: Jun. 2, 2011

(54) Dynamic hallux valgus corrector

(75) Inventor: Daniel Rafique, Woodbridge (CA)

(73) Assignee: DANIEL RAFIQUE, Woodbridge (CA)

(21) Appl. No.: 12/926,641

(22) Filed: Dec. 1, 2010

(30) Foreign Application Priority Data
Dec. 1, 2009 (CA) 2686487

(51) Int. Cl. A61F 5/14

(52) U.S. Cl. 602/30

(57) ABSTRACT

The invention corrects the dynamic mechanical dysfunction of the hallux valgus toe during sports-specific gaits and weight-bearing activity with an orthotic featuring a toe-supporting structural band that inserts into the user's existing shoe, thereby increasing stability in the structure of the foot and lower limb. Correcting this instability as the transverse and medial longitudinal arches buckle and the knee rolls medially amplifies the recruitment of muscle fibers chronically deactivated in the hallux valgus gait, namely the muscles of the inside lower limb and the posterior chain. The invention enables greater sports-specific speed, balance, agility, endurance, power delivery through the foot into the ground, stability of the lower limb (and therefore the entire body) as it engages surfaces, and corrects muscular imbalances by re-training neuromuscular activation patterns. The construction technique of this invention is applicable to correct hammertoes, overlapping toes, tailor's bunion, and may reduce pain associated with bunions.

### BACKGROUND

1. Field

The present invention primarily relates to an orthotic device of the foot that corrects hallux valgus and the dysfunctional hallux valgus gait in sports-specific activity, general walking, and weight-bearing movements, but provides as an optional feature through its unique constructional technique corrective support to the malformation and malfunction of any of the other toes (overlapping toes, hammertoes, and tailor's bunion).

2. Prior Art

Previous devices designed to correct hallux valgus have been designed as autonomous units such as splints or pads attachable to the toes, foot or ankle to be used statically outside of a shoe. The disadvantage of these devices is that none of them can be used dynamically, as in running or skating, for example; the weakened muscles of the hallux valgus foot cannot be trained or strengthened. (U.S. Pat. Nos. 4,644,940, 7,04642, 361959)

Some devices have been designed with the purpose of alleviating pain associated with bunions. These have not been specifically designed to or claim to correct the dysfunctional gait inherent in the lower limb with hallux valgus present. Hallux valgus is not always associated with pain. (U.S. Pat. Nos. 5,282,782, 262334)

The construction of previous devices have been bulky, with intricate mechanics or moving hinges, springs and parts, heavy, constructed of inflexible materials such as metal, wood or plastic which have been impractical and unsafe for use within a user's existing shoe, especially during rigorous, aggressive sports-specific activity. (U.S. Pat. No. 4,244,359, U.S. Pat. No. 3,049,120, U.S. Pat. No. 4,729,369, CA1126604, CA1167639)

Previous devices such as splints which rely on their attachment to the foot or ankle cannot generate enough mechanical advantage to satisfactorily pull and stabilize the hallux toe away from the lateral side of the foot, especially while in a shoe, or during aggressive sports type gaits or weight-bearing activity such as weight training, as in the seated leg-press or squat. (U.S. Pat. No. 4,644,940, U.S. Pat. No. 352115, U.S. Pat. No. 5,282,782, U.S. Pat. Nos. 5,437,616, 393834, U.S. Pat. No. 4,644,940) (U.S. Pat. No. 6,318,373)

### ADVANTAGES

Accordingly, several advantages of one or more aspects are as follows: to provide stability to the structures of the human foot that activate the recruitment of propulsive muscles by providing enough mechanical leverage to dynamically rectify the location of the hallux in the hallux valgus foot and gait; especially during rigorous sports-specific gaits or movements with an apparatus that is durable, flexible, simple, safe, light, adjustable, available in an array of shapes and sizes, and that can readily fit into users' existing footwear. Providing stability to the foot, and rectifying and enhancing the propulsive action of the hallux improves an athlete's overall stability, power, agility, and endurance. Another advantage is that the recruitment or activation of muscle fibers consequently deactivated, weakened or made dormant by hallux valgus, subsequently become activated; over time, the brain-body learns to fire these muscles regularly. Other advantages of one or more aspects will become apparent by considering the drawings and description that follow.

### SUMMARY OF THE INVENTION

In accordance with one embodiment, a human foot device for repositioning the hallux comprises a platform of a shape to receive the sole of a human foot, a band to support the hallux, especially during gait or propulsion, and definable adjustments and various methods of affixing the band to the platform. In some embodiments, the platform is further made of a structural base and a covering.

### BRIEF DESCRIPTION OF THE DRAWINGS—FIGURES

FIG. 1 is a view of the preferred embodiments applicable to rigid-soled footwear for a right foot from above.

FIG. 2 is a side view of the same embodiments as seen from the hallux side

FIG. 3 is a side/partial top view of the same embodiments as seen from the fifth toe side.

FIG. 4 is a view of the same embodiments from below showing the variability of the band's attachment location.

FIG. 5 is a view of the same embodiments from below with the band removed.

FIG. 6 is a view of a right foot with hallux valgus from above.

FIG. 7 is a rear view of the same foot in FIG. 6.

FIG. 8 is a top view of a right foot with the embodiments being worn.

FIG. 9 is a rear view of the same foot with the same embodiments being worn.

FIG. 10 is a perspective view of the embodiments for rigid-soled footwear being applied to the foot.

FIG. 11 is a perspective view of the same embodiments sliding into a shoe.

FIG. 12 is a top view of embodiments adapted specifically to align hallux valgus, overlapping toes, hammer toes, and tailor's bunion.

FIG. 13 is the cross-sectional view x-x the heel cup as the embodiments relate to a running shoe.

FIG. 14 is a top view of the embodiments adapted specifically to flexible-soled footwear with no structural base or secondary band.

FIG. 15 is a perspective view of the same embodiments from below showing stitchery and a slit.

FIG. 16 shows the same embodiments rolled up within a clenched fist.

FIGS. 17 to 17A is a top view of embodiments adapted to flexible-soled footwear showing a secondary band attached to the first band, with the band in open and closed positions.

FIG. 18 shows the same embodiments worn in a shoe with the user pulling the main band over the first metatarsophalangeal joint by grasping the secondary band.

FIGS. 19 to 19A shows the base of a platform with covering removed having adjustable band attachment.

FIG. 20 shows the base of a platform with covering removed having an alternate method of band attachment.

FIGS. 21 to 21A shows the base of a platform with covering removed having adjustable band attachment; and shows slit locations.

FIG. 22 is a bottom view of a skeletal foot with hallux valgus showing the dysfunctional, inefficient curved path of contracting force of the flexor hallucis longus tendon.

FIG. 23 are graphs of 3d gait analysis of a cyclist with hallux valgus present in the right foot riding a bicycle

FIG. 24 shows a perspective view from above of embodiments with adjustable length platform.

### DETAILED DESCRIPTION OF THE PREFERRED EMBODIMENTS

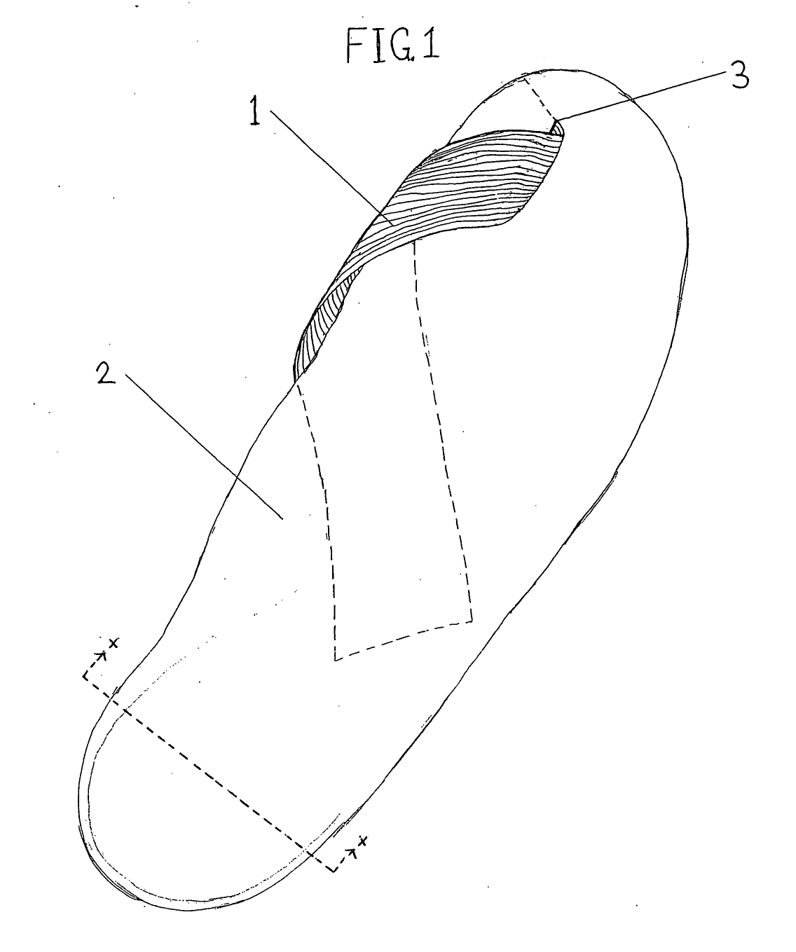

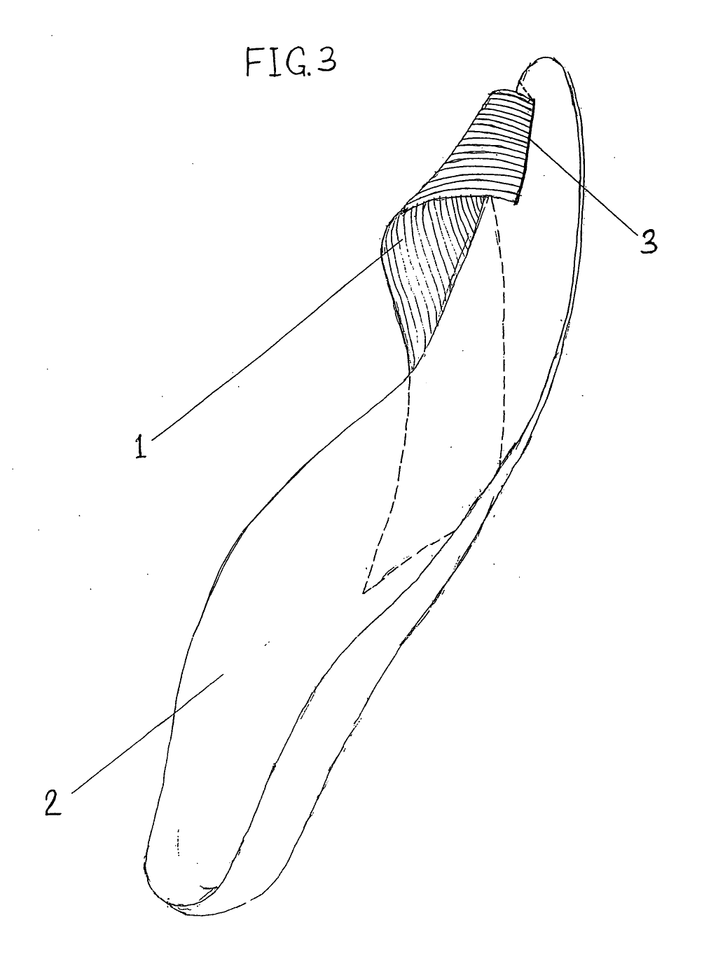

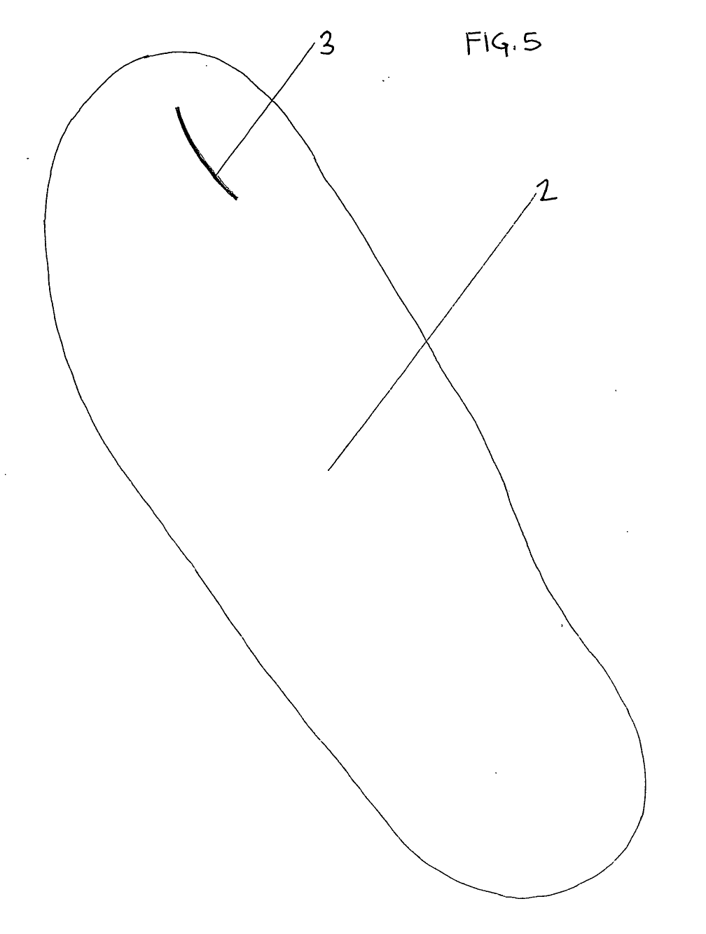

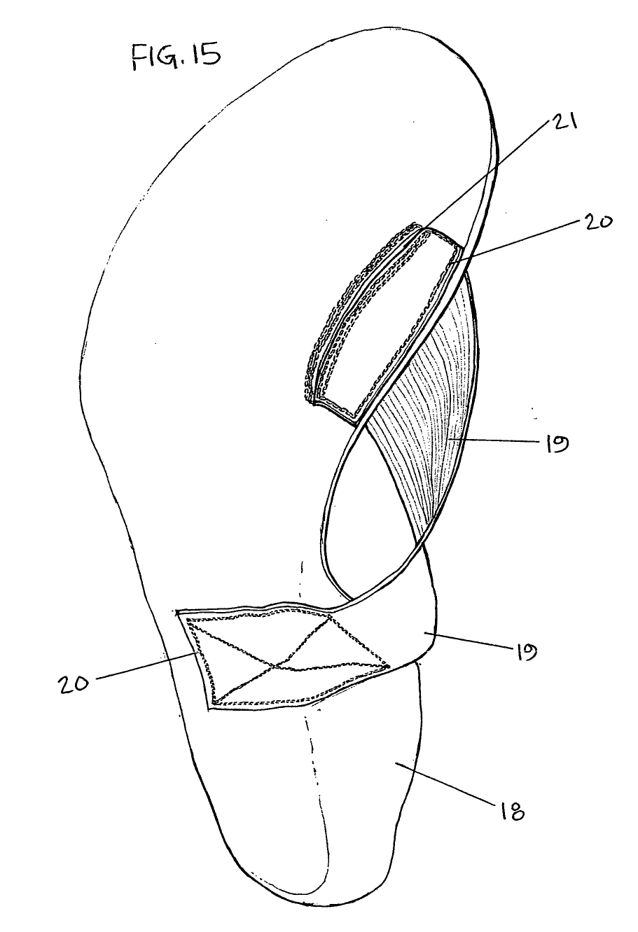

Two sets of embodiments each having separate constructional methodologies and materials, and applications are described. The two types are: 1. Embodiments for flexible-soled footwear (FIGS. 14 to 18) and, 2. Embodiments for rigid-soled footwear (FIGS. 1 to 5).

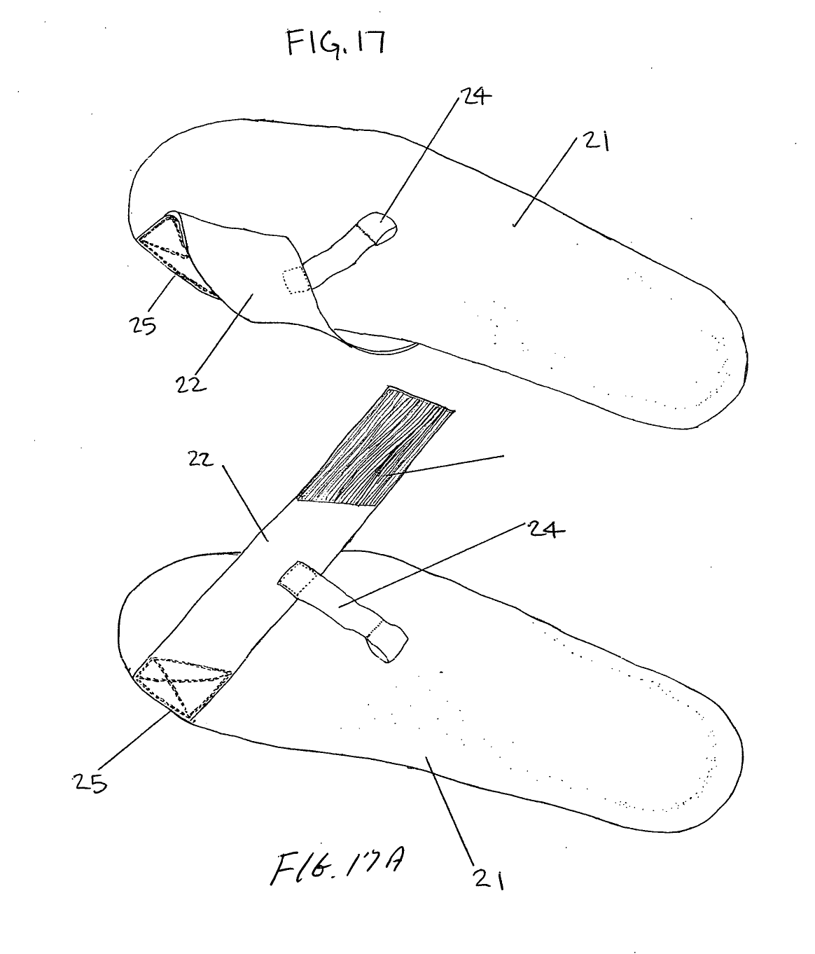

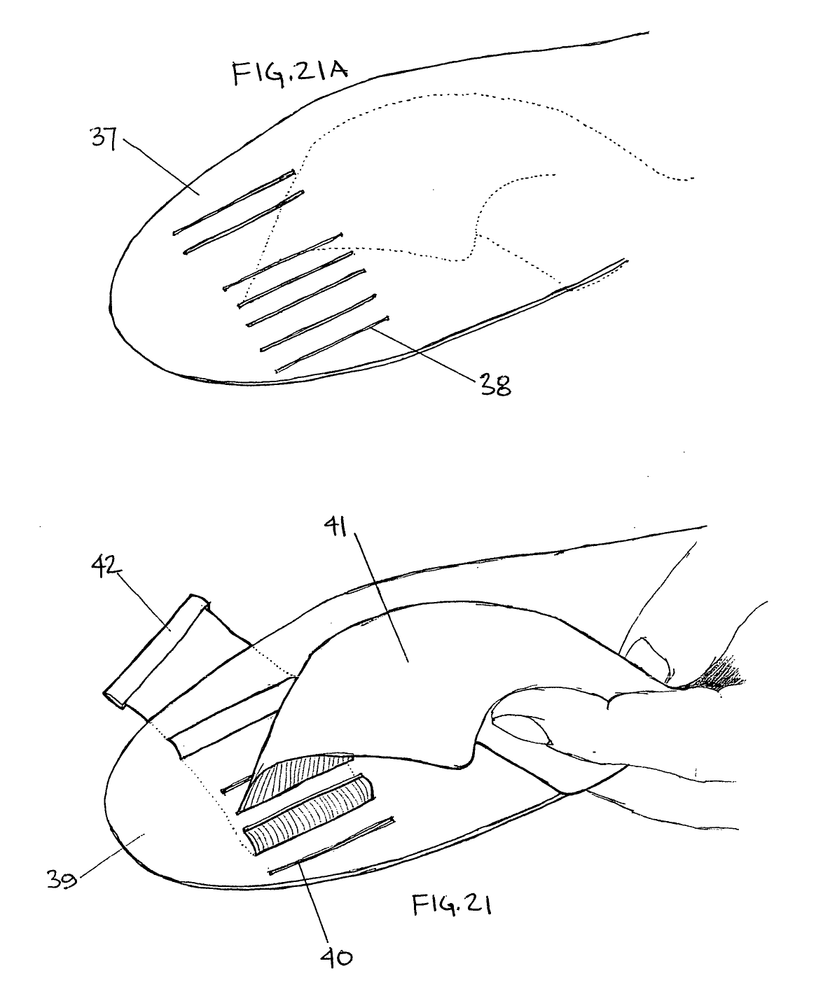

Further to these two applications, each type of embodiment can either be: A. Built custom by a trained professional to suit a particular user's foot in their existing footwear (FIGS. 1 to 5; FIGS. 14 to 16), or B. Purchased ‘off-the-shelf’ with adjustable modifications definable by the user to suit their feet and footwear (FIG. 17 to FIG. 21). Some of the constructional methodologies and materials are interchangeable across applications.

Without specific reference to the various constructional methodologies, all embodiments for both applications have a platform the full length of a foot. Attached to the platform is a structurally woven band. The platform acts as a structural member, or grounding, to the band. The band has two attachment points to the platform and various methods for attachment; some attachment methods are fixed permanently while others offer a range of adjustment to the band's attachment location in relationship to the platform. The band's origin attaches to the platform beneath the hallux, passes upward between the hallux and index toe, wraps around the hallux and the first metatarsophalangeal joint, thereby pulling the hallux distal from the index toe into a corrected position; the band's terminus reattaches to the platform under the area of the longitudinal medial arch. The embodiments of some platforms are made by joining a structural base to a soft covering. The underside of the platform has an optional method for fixation to the inside of the footwear, especially for the application of flexible-soled footwear; Paper-thin double-sided adhesive tape or ultra thin hook and loop fastener can be used, but alternative methods may be used. The preferred embodiments, once worn and inserted into appropriately sized footwear work in unison, giving the entire assembly (that is foot, band, platform, and shoe) further structural capacity to reinforce the repositioning of the hallux during gait, standing, or weight-bearing pushing movements such as those in weight training.

Embodiments for the application of rigid-soled footwear are shown in FIGS. 1, 4, 5 and 8. These embodiments can be constructed with materials exhibiting little or no flexibility. The platform 2 is constructed of material resistant to compressive forces in the vertical plane and torsional forces in the lateral plane that resist flexion along its longitudinal axis. Since there is no or little flexion in rigid-soled footwear, there will be little or no flexion at the first metatarsophelangeal joint and therefore the structurally woven band 1 may be constructed of an inelastic material, such as rubber-backed vinyl belting material, woven nylon, structural strapping, or other suitable materials. In this case, the band does not stretch along its longitudinal axis but is bendable across its width to wrap and conform to the hallux and first metatarsophelangeal joint. Band 1 is paper thin or thinner than a few millimeters, and the width of band 1 may be variable along its length. Alternatively, band 1 may also exhibit elastic properties, incorporating fibers exhibiting elastic properties into the weave. Rigid acrylic composites, thermoplastics, or carbon fiber can be used for the base 2, but alternative materials may also be appropriate. A soft covering of dense foam, medical-brand cellular urethanes, synthetic or natural leather, or vinyl can be used as covering to base 2.

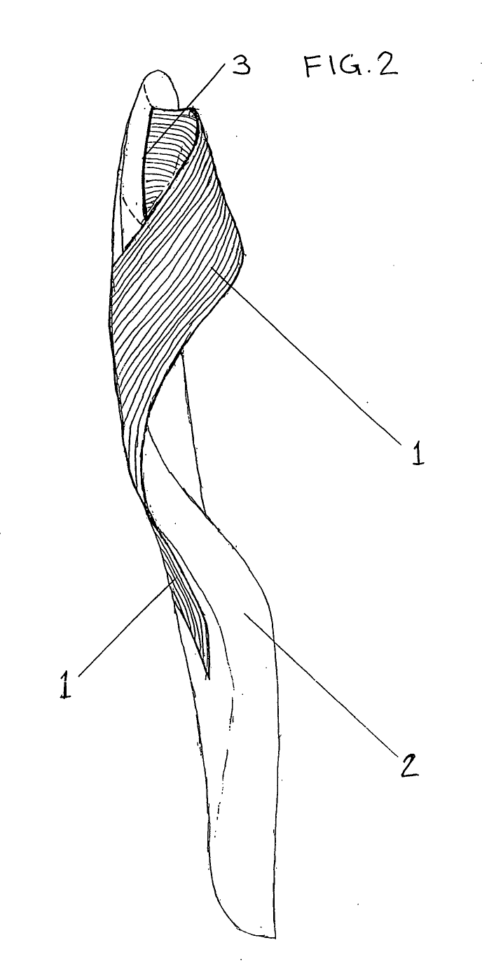

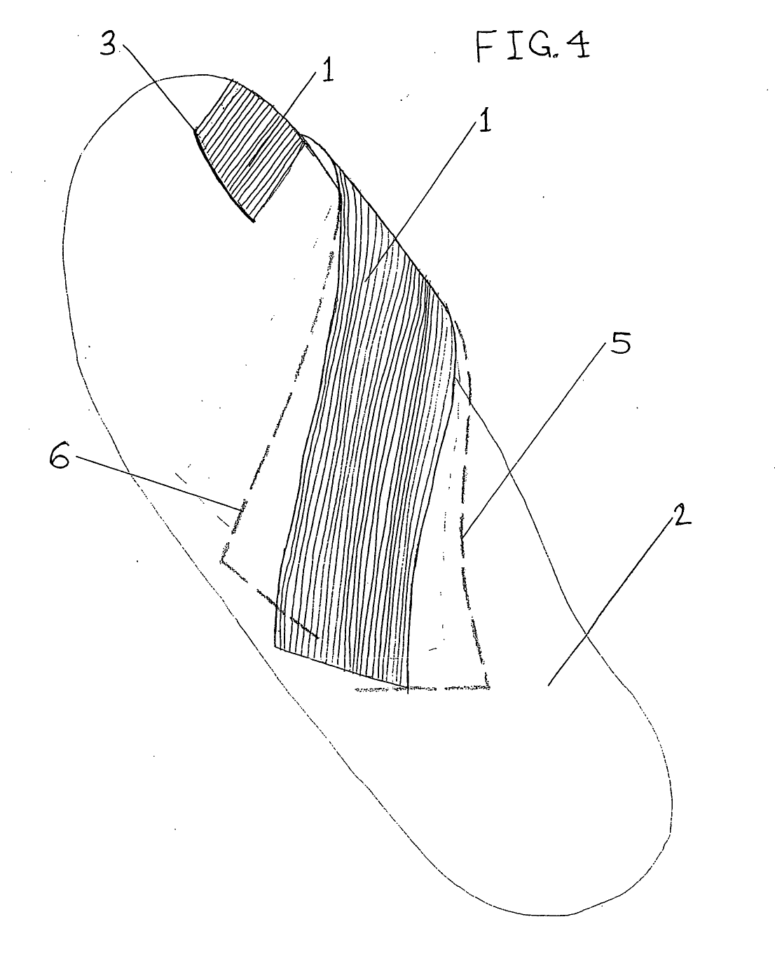

With reference to the drawings, specifically FIGS. 1, 4, 5 and 8, the embodiments shown are adapted to rigid-soled footwear. The structural band 1 for the hallux 4 is first fastened to the undersurface of the platform 2, that then emanates through a slit 3, located between the hallux and index toe, corkscrewing or looping over and across the hallux 4, thereby pulling it distal, away from the index toe and supporting it in place. After the band 1 passes over the hallux, it is then fastened to the undersurface of the platform 2 once again (FIG. 4). The entire length of the structural band forms a ribbon-like looping ‘corkscrew’ formation (FIG. 2) throughout its passage from under the platform 2, over the hallux 4, and back under the platform 2 (FIG. 1 to FIG. 4).

With reference to FIG. 4, Band 1 and can be re-fastened either permanently with a bonding adhesive or with hook and loop closure where it re-attaches to the undersurface of the platform 2 under the area of the ball of the foot extending backward toward the medial longitudinal arch as indicated by the dashed lines. The location of where the band 1 is re-attached to the undersurface of platform 2 is variable as indicated by the hatched lines 5 and 6.

With reference to FIG. 4, for the application of rigid-soled footwear, since the foot and shoe will not flex, the centerline of band 1 passes over the junction of the first metatarsal and first proximal phalange before it is fastened once again at location 5 to the underside of platform 2.

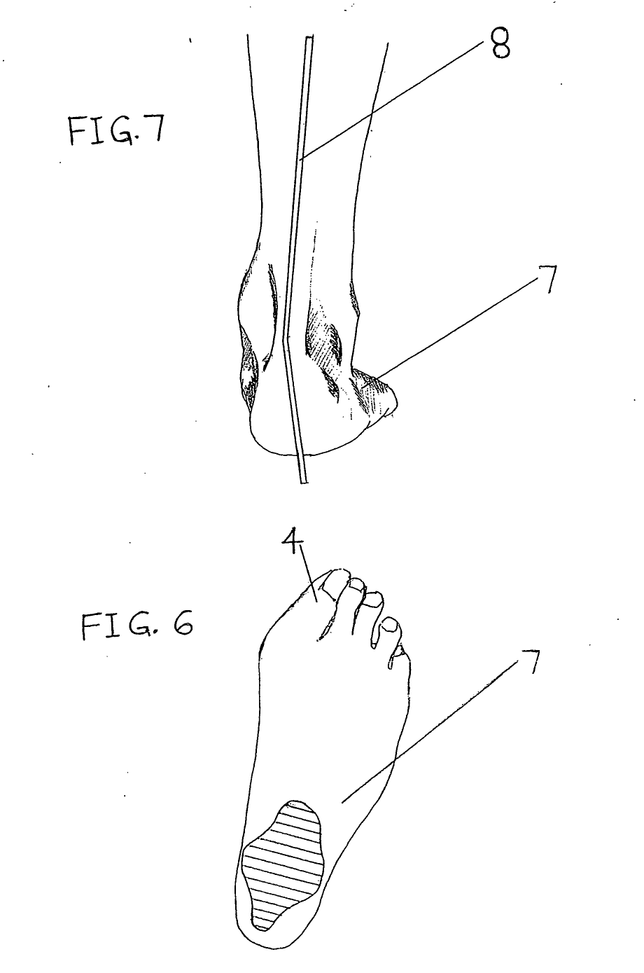

With reference to FIGS. 6 and 7, hallux 4 of the foot 7 exhibits hallux valgus. FIG. 7 shows buckling of the transverse and medial longitudinal arches causing a pronated position of the subtalar joint as indicated by the marker 8. This foot is an unstable structure.

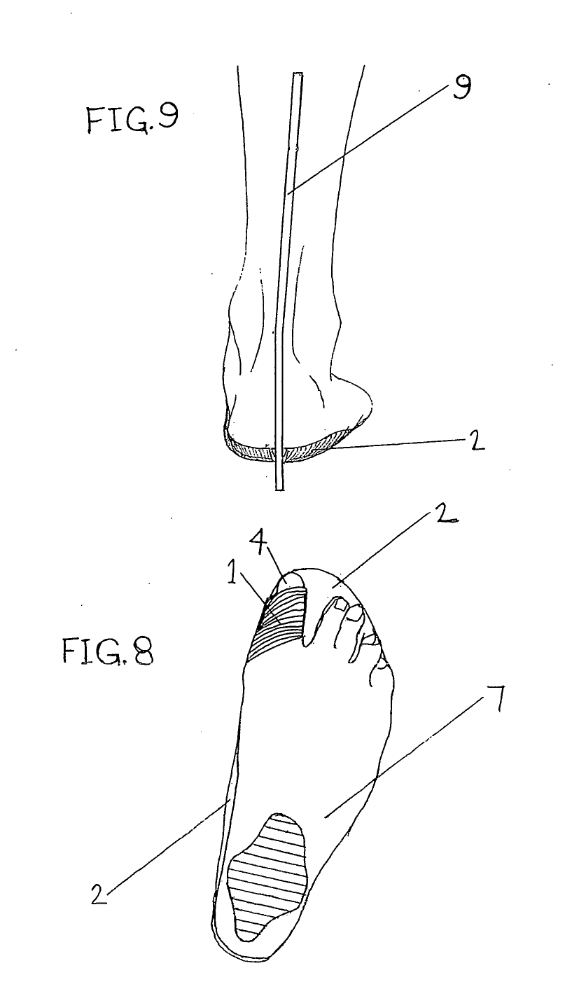

With reference to FIGS. 8 and 9, greater neutral position of the subtalar joint is indicated by the marker 9 as the embodiments are being worn by the foot 7, thereby amplifying support of the transverse and medial longitudinal arches by correcting the location hallux 4.

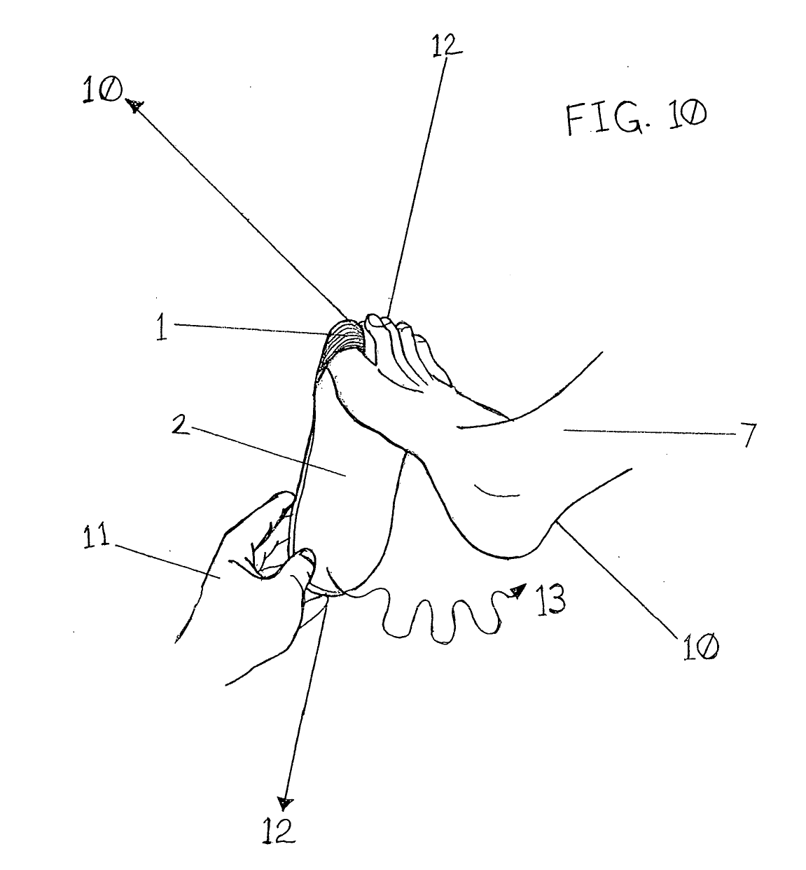

With reference to FIG. 10 showing embodiments for rigid-soled footwear with permanently affixed band 1, the right foot 7 is forced into the tight opening of the band 1 along the trajectory 10 while platform 2 is being pulled by the left hand 11 along the trajectory 12 while simultaneously wiggling platform 2 along a rotational trajectory 13 which binds hallux 4 into place.

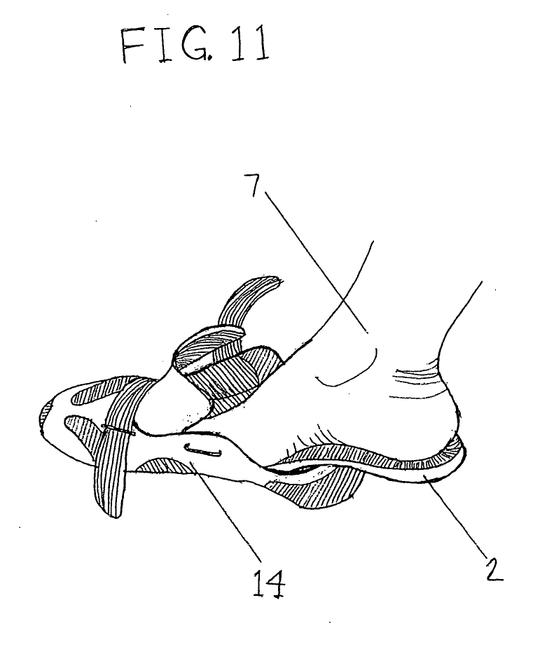

With reference to FIG. 11, while wearing platform 2, foot 7 slips into the footwear 12. Note that platform 2 dangles away from the heel of foot 7 but once foot 7 and the embodiments are in footwear 12 and footwear 12 is laced or done up, foot 7 and platform 2 fit together as though the embodiments were in the footwear 12 before installing foot. The structure of band 1 is enhanced as it is compressed between the mid-sole of footwear 12 and the undersurface of platform 2 while foot 7 bears weight in footwear 12.

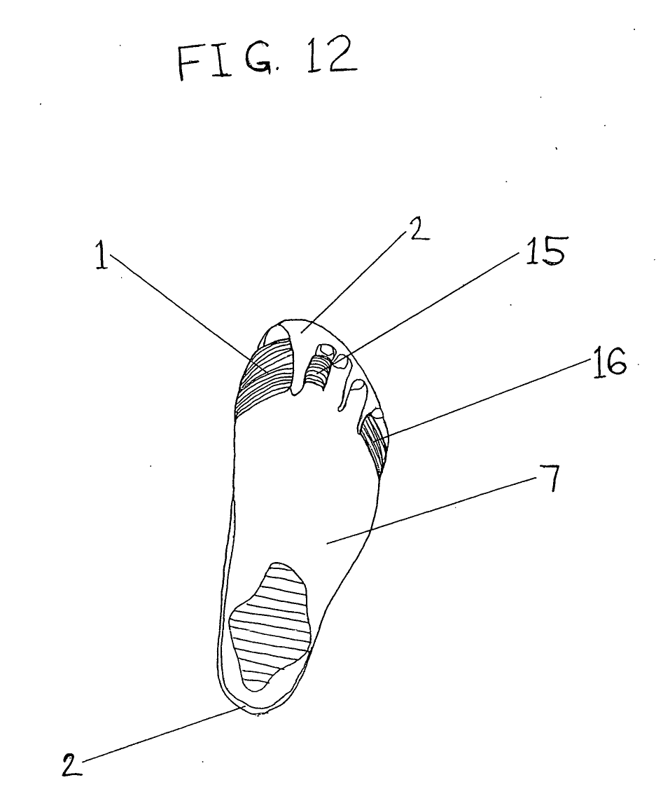

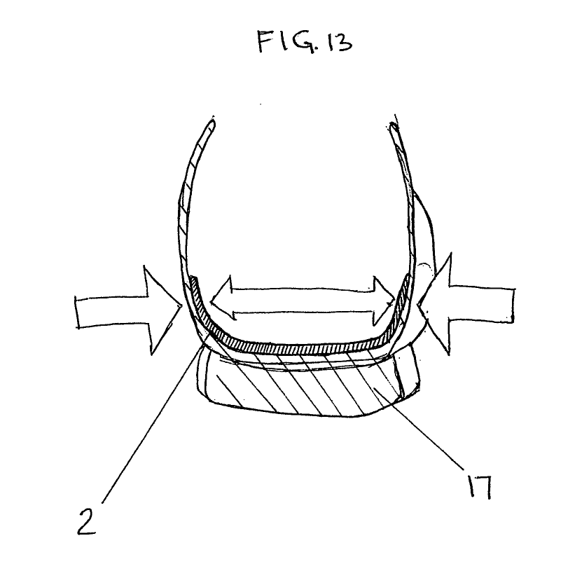

With reference to FIG. 12, the band 15 supports the index digit with hammertoe, or overlapping toe biomechanical deficiencies. The band 16 is for tailor's bunion, and is of identical structure and assembly of band 1 except that it is mirrored and smaller in scale to adapt to the smallest toe. Band 15 is adhered at either end to the undersurface of platform 2 where platform 2 accomodates band 15 through slits cut into platform 2 at either end of the disfuctional index toe.

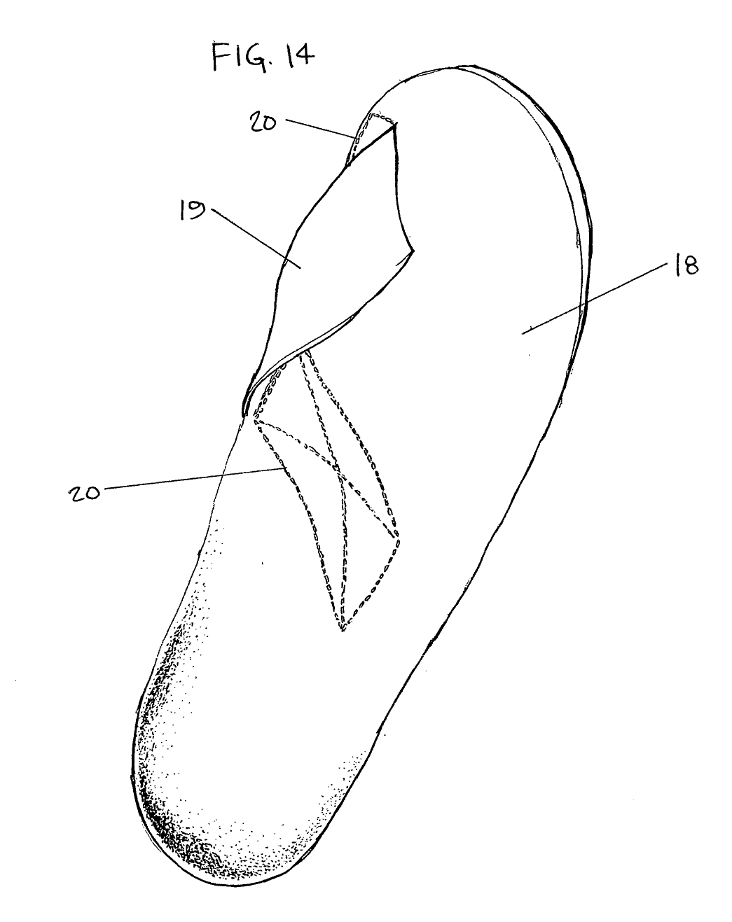

With reference to FIG. 13, the sectional view x-x is shown. Platform 2 has a heel cup bilaterally encircling the heel. All versions of the embodiments' platforms can have a heel cup of various depths. One function of this embodiment is to provide stability to the heel and rear-foot as it relates to the platform. A second, more important function of a heel cup, is to further enhance the platform's structural relationship to the footwear 17. The edges of the heel cup encircling the heel portion of the platform inhibit lateral movement of the platform within footwear 17 by pressing against the interior vertical walls of footwear 17 that correspond to the heel cup of the footwear.

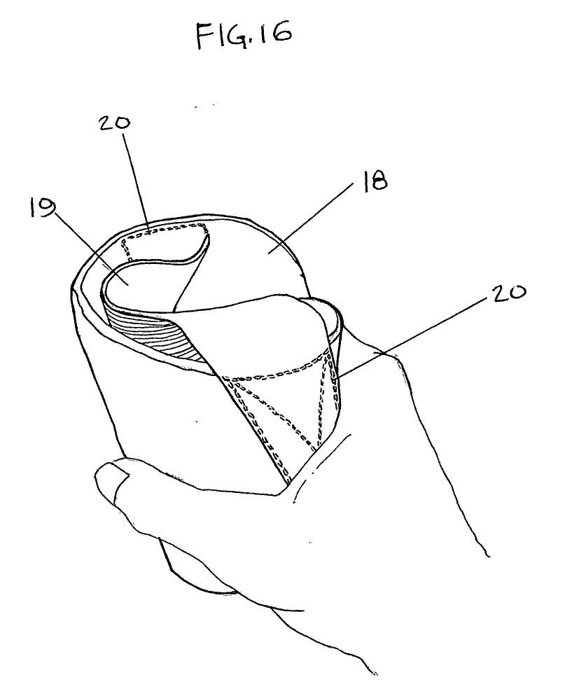

With reference to FIGS. 14 to 16, embodiments for flexible-soled footwear are shown. Solely flexible materials such as high-density foam (EVA), medical-brand cellular urethanes can be used for the platform 18. The band 19 can be stitched, as with a sewing machine, fused, or adhered in order to permanently fix the band to either side of the platform, and at either end of the band, with or without the band passing through any slit in the body of the platform. The embodiment shown has the band 18 stitched 20 to the undersurface of platform 18 where band 19 then passes through a slit 21 [FIG. 15]. Band 19 is subsequently affixed by stitchery 20 to platform 18 under the region of the longitudinal medial arch [FIG. 15]. The terminal location of band 19 can be affixed such that the centerline of band 19's width is fore of the junction of the first metatarsal and first proximal phalange. This adjustment provides a definable amount of lateral pressure from band 19 onto the first metatarsophelangeal joint from band 19, and further defines the amount of hallux abduction. With reference to FIG. 16, the embodiment is rolled up in a clenched fist exhibiting its superior durability, simplicity and flexibility. This embodiment, with no structural base joined to the undersurface of platform 18 can be affixed to the inside of the users footwear with a paper-thin double-sided adhesive, or thin hook-and-loop fastening.

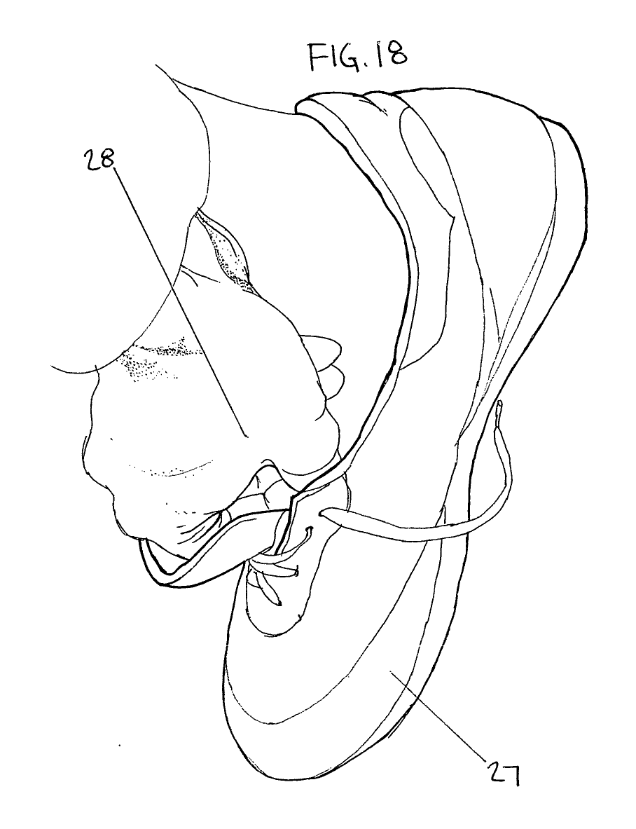

With reference to FIGS. 17 to 18, embodiments for flexible-soled footwear are shown. The band 22 is affixed permanently to the platform 21 with stitchery 25. The terminus of band 22 has hook and loop closure mechanism 23 stictched, adhered or fused to it. FIG. 17 shows band 22 undone from platform 21. a secondary band 24 is fused, adhered, or attached by stitchery to band 22. A secondary band 24 is used for pulling 28 [FIG. 18] band 22 over the first metatarsophalangeal joint once the embodiment is worn and placed in the footwear 27. Platform 21 is further adhered to the inside (mid-sole) of footwear 27 with a double-sided adhesive or thin-hook-and-loop fastening. Adhering platform 21 to footwear 27 in this fashion inhibits band 22 from rising between the toes; Essentially, the structure inherent in the mid-sole of the footwear acts as the structural base to the flexible platform.

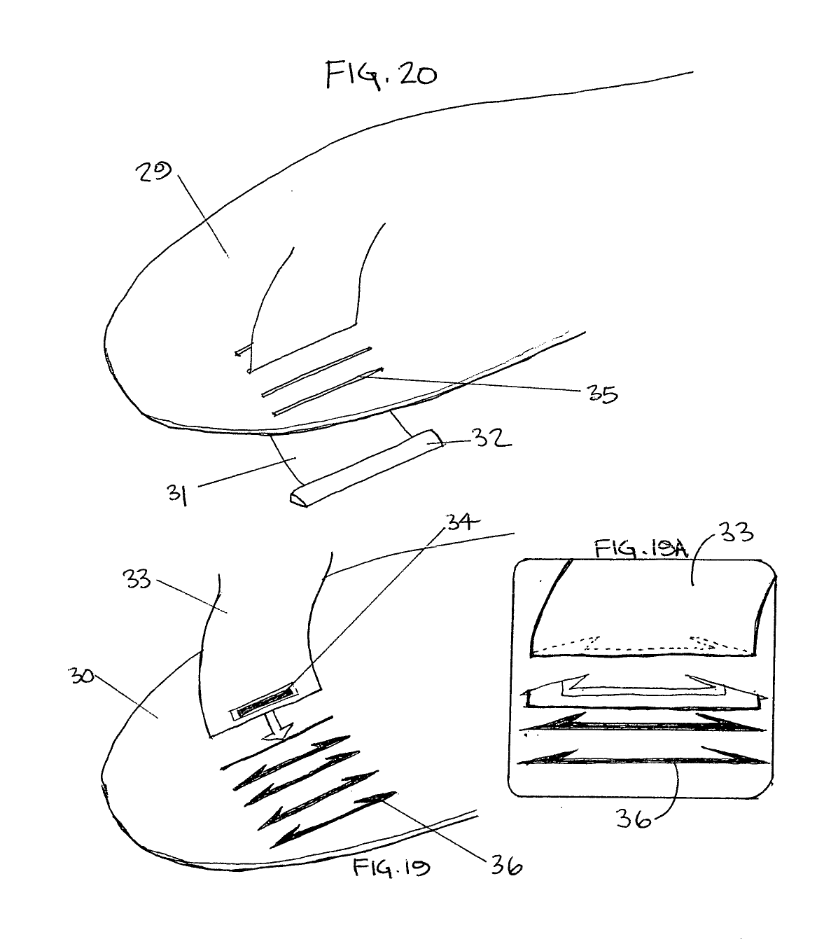

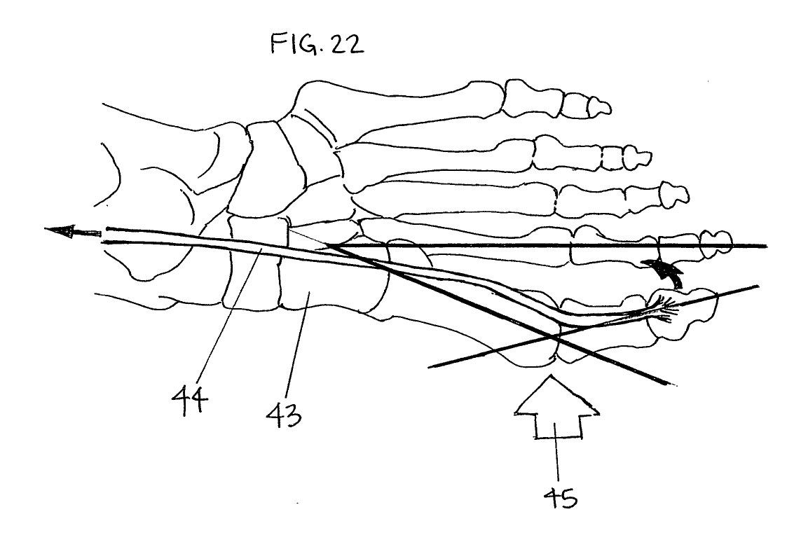

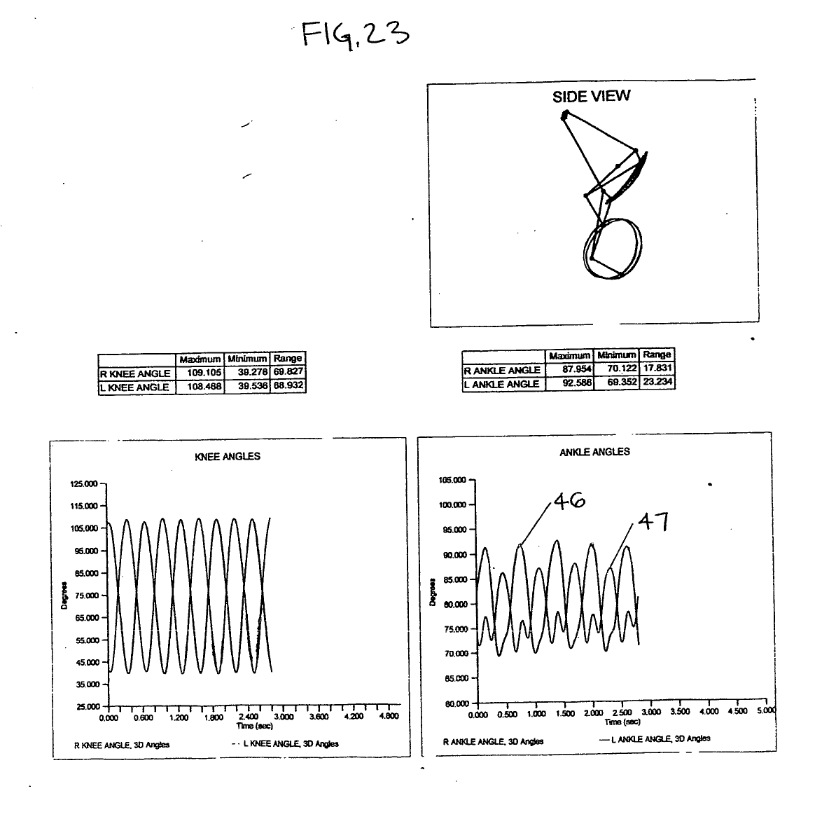

With reference to FIGS. 19 to 22, embodiments for flexible-soled applications have either a structural base joined to a soft covering, or made with no structural base (embodiments adhered to the inside of the user's shoe). FIGS. 19 to 21A show various methods for affixing the origin of the hallux-supporting band 31 and 33 to the structural base 29 and 30 when a base is present. Joining a structural base to a soft covering precludes the adhesive tape fastening to the undersurface of a platform to the inside of the footwear, although these embodiments might also be used with such adhesive to further enhance the platform's relationship to the footwear. Each method of band affixation describes definable adjustments to fit the position of users' hallux. Adjusting the location of the band's origin defines the amount of force the band applies to the hallux as it pulls it away toward the medial side of the foot; and how much lateral pressure the band is applying to the first metatarsophalageal joint 45 [FIG. 22]. These adjustments define the degree to which the acute interior angle inscribed by the first and second metatarsals is decreased, and the degree to which the obtuse interior angle inscribed by the first proximal phalange and said first metatarsal is increased. New users should start with less hallux deflection since adaptation of the tendons and muscles must progress slowly.

The structural base can made with materials that are flexible in its longitudinal axis so as to conform with flexion at the 1^{st} metatarsophalangeal joint, yet sufficiently rigid in the lateral axis to ground or anchor the band. Semi-rigid high density foam, thermoplastic composites, or plastics for base can be used, but alternative materials might also be used.

With reference to FIG. 22, the bottom view of a skeletal foot with hallux valgus 43 showing the dysfunctional, inefficient curved path of contracting force of the flexor hallucis longus tendon 44. As the flexor hallucis longus muscle is fired and contracted, the tendon 44 applies a pulling force to the hallux that causes the hallux to adduct laterally instead of asserting leverage and purchase into the surface it engages. This lateral movement of the hallux further compromises the structures of the foot thereby inhibiting the recruitment of the propulsive muscles of the limb from firing or activating at the correct time during gait. Namely, these muscles or fibers of these muscles are the flexor hallicus longus, the lateral and medial heads of the flexor brevis, the aductor hallicus, flexor digitorum longus, muscles of the gastrocnemius (especially the medial head), semimembranosus, semitendinous, vastus medialis of the quadriceps, the Sartorius, the adductors of the thigh, and also the powerful posterior chain (gluteus maximus, hamstrings, and lower back muscles). Wearing the preferred embodiments amplifies the recruitment of muscle fibers chronically neurologically deactivated, weakened, dormant, or unable to forcefully assert purchase onto the surface that the foot is pushing off of in the hallux valgus foot and leg. The embodiment also activate the propulsive muscles earlier in the gait cycle since the hallux, in its corrected position receives information from the surface it engages on when to activate, thereby activating other muscles in the gait sequence earlier. The invention might also enhance the activation of other muscle fibers not mentioned above.

Correcting the location of the hallux valgus toe and firmly supporting it in place provides greater support to the structures of the transverse and longitudinal medial arches of the foot, enabling a greater neutral position of the subtalar joint, preventing the ankle and knee to rotate and ‘roll’ or ‘knock’ inward. The biomechanical deficiency of the hallux valgus toe has the same affects on the deactivation patters of the neuromuscular response both in gait cycles where the hallux toe pushes and rolls off the ground with flexion at the first metatarsal/first proximal phalange, and also in pushing motions such as bicycling with rigid-soled shoes, or squatting weight overhead where there is no flexion at the first metatarsal/first proximal phalange. Wearing the preferred embodiments amplifies the recruitment of muscle fibers, and enhances the support of the structures of the foot and lower limb in both rigid-soled and flexible-soled footwear.

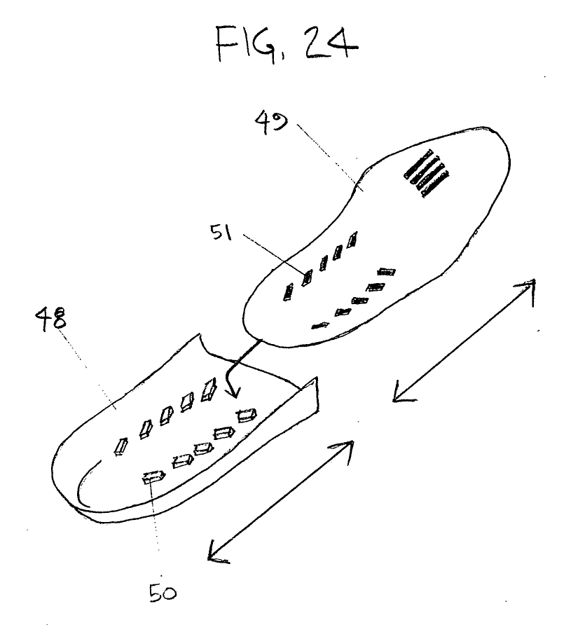

FIG. 23 shows graphical representations of data collected on the 3Demensional gait analysis of cyclist riding a bicycle at approximately 100 RPM. 3D markers are placed on the cyclists bony landmarks, the knee, ankle, hallux, and hip; infra-red cameras record the cyclists gait. Graphs are shown plotting angles of the knee and ankle for both legs. The graph ANKLE ANGLES plots a range of ankle angles along the vertical axis of the graph, and time along the bottom axis of the graph. The significance of time in this context is that it relates to the rotational phases of the bicycle crank arms (power phase as with the leading leg, recovery phase as with the trailing leg, dead top center, and dead bottom center). Line 46 is the plotted data of ankle angles for the left leg; Line 47 is the plotted angle for the right leg. The right leg has hallux valgus, where the left leg does not. It is visually clear through the graphic representation that the legs' pedaling action, particularly in the ankles, are not symmetrical. As the rider approaches a horizontal crank arm, 3 O'clock, with the leading, propulsive (right) leg (the left leg is in recover phase with the cranks coming around from 9 o'clock), the dysfunctional hallux moves laterally toward the baby toe instead of asserting leverage and purchase into the ground (in this case, the surface of the sole of the shoe). The structures of the foot that normally operate to transfer load into the ground from the associated limb and propulsive muscles consequently collapse. This collapse occurs on each pedal stroke, since in effect, the pedal is the surface receiving the load from the leg. The rider is unable to transfer force from the muscle groups of the leg into the pedal through the sole of the shoe. As the structure of the foot collapses, the affect is that the pedal falls away from the foot at each pedal stroke at the moment the rider tries to engage the pedal with force such that the rider is constantly trying to reach the pedal as it falls away from the foot by tip-toeing with a plantar flexed ankle.

### Additional and Alternative Embodiments

In addition to correcting the dynamic dysfunction of the M4 toe in users who were born or developed the Hallux Valgus condition, the invention can also successfully correct an ‘Effective Hallux Valgus Condition’ found in people who exhibit no signs of Hallux Valgus. Some shoes have a narrow or pointed toe box which users unknowingly create an ‘Effective Hallux Valgus Condition’ by squashing the hallux toe toward the little toe by wearing such shoes. Stuffing the foot into such a shoe effectively creates a condition similar or identical to users with an intrinsic Hallux Valgus condition. The present invention inhibits ‘Effective Hallux Valgus Condition’ in all shoes but the user may have to purchase new shoes with a toe-box that can accommodate the corrected position of the hallux toe that the invention provides.

The construction technique of this invention is applicable to correct hammertoes, overlapping toes, tailor's bunion, and may reduce pain associated with bunions. Any number of structural bands of various sizes and widths with any number of slits located between any of the toes can be incorporated into the PLATFORM to correct malformations and biomechanical deficiencies of any

The invention can be used without socks in sporting applications or with ‘five-finger’ socks such as those designed by Vibram (Damon Mill Square #H3 Concord, Mass. 01742 , 978.318.0000) called ‘injinji’. ‘Five finger’ type socks are available by other manufacturers as well.

What is claimed is:

1. A human foot device for repositioning the hallux comprising:
a. a platform of a shape to receive at least a sufficient portion of the sole of a human foot, said platform has an upper surface, an undersurface under said upper surface, a forefoot portion, and a heel portion behind said forefoot portion,
b. a band of a predetermined length and a sufficient width to support said hallux, said band has an origin, a terminus, and an intermediate portion between said origin and said terminus, and
c. at least one means of affixation at said band's origin and at least one means of affixation at said band's terminus to at least one surface of said platform; said band's origin first is affixed under the approximate region of the forefoot, second said band's intermediate portion passes over said hallux toward the approximate area of the first metatarsophalangeal joint of said foot in a corkscrew wraparound formation, third said band's terminus is affixed under the region encompassing the approximate area of the tip of said hallux to the approximate area of the medial longitudinal arch of said foot so as to be able to reposition the hallux whereby it deviates from the rest of the toes.

2. The device recited in claim 1 wherein said platform further comprises:
a. a base of at least one layer of a material of sufficient rigidity so as to anchor said band's affixation to said platform,
b. a covering of at least one layer of a material of sufficient malleable density so as to conform to said sole of human foot, and
c. means for joining said base to said covering.

3. The device recited in claim 1 wherein a secondary band of a predetermined length is attached by said secondary band's end to said band between said band's origin and said band's terminus at a sufficiently blunt angle.

4. The device recited in claim 1 wherein said platform's undersurface has at least one means to join said platform to the inside of a body for encompassing feet.

5. The device recited in claim 1 wherein said platform further comprises at least one slit, said slit's location is in the approximate region between the medial extent of said hallux in said hallux' repositioned location and the lateral extent of the fifth toe as said foot relates to said platform, said slit runs approximately parallel to said index toe, said slit is of sufficient size to receive said band; said band's origin first is affixed by at least one means of affixation to said platform's undersurface, second said band's intermediate portion passes through said slit before, third said band's intermediate portion travels upward between said hallux and said index toe.

6. The device recited in claim 1 wherein at least a section of said heel portion of said platform further comprises a raised edge sufficiently curved upward from said platform's upper surface, said raised edge passes around the extent of the heel portion so as to sufficiently cup the heel of said human foot, whereby said raised edge inhibits lateral movement of said platform within said body for encompassing feet.

7. The device recited in claim 2 wherein said base further comprises at least one slit, said slit's location is in the approximate region between the medial extent of said hallux in said hallux' repositioned location and the lateral extent of said fifth toe as said foot relates to said platform, said slit runs approximately parallel to said index toe, said slit is of sufficient size to receive said band; said band's origin first is affixed to said platform's underside where, second said band's intermediate portion passes through said slit before, third said band's intermediate portion travels upward between said hallux and said index toe.

8. The device recited in claim 1 wherein said platform is rigid.

9. The device recited in claim 1 wherein said platform is flexible.

10. A method of improving the propulsion sequence of the human gait comprising:
b. applying an effective amount of force to reposition the hallux such that the acute interior angle inscribed by the first and second metatarsals is decreased, and the obtuse interior angle inscribed by the first proximal phalange and said first metatarsal is increased whereby lateral adduction of said hallux is restricted.
b. straightening the path of contracting force of the flexor hallucis longus tendon such that said asserts purchase and leverage onto the surface said hallux engages.
c. reducing the leverage of the transverse head of the adductor hallucis and the oblique head of said adductor hallucis on the first proximal phalanx thereby balancing the leverage of the abductor hallucis and the medial tendon of the flexor hallucis brevis is achieve.
d. training the propulsive muscles of the foot and limb to activate earlier in gait and with greater force.

11. The method recited in claim 10 wherein said improving the propulsion sequence of the human gait further comprises enwrapping said hallux with a band of sufficient width and predetermined length, said band is affixed to a platform of a sufficient shape to accommodate at least a substantial portion of a human foot

12. The method of claim 11 wherein said improving the propulsion sequence of the human gait further comprises fitting said enwrapped hallux with said foot touching said platform into a body for encompassing feet.
